(19) United States
(12) Patent Application Publication
(10) Pub. No.: US 2023/0081269 A1
(43) Pub. Date: Mar. 16, 2023

(54) Barrel Clamping Mechanisms, Systems, and Methods for 3D Printing

(71) Applicant: Advanced Solutions Life Sciences, LLC, Louisville, KY (US)

(72) Inventor: Scott Cambron, Louisville, KY (US)

(73) Assignee: Advanced Solutions Life Sciences, LLC, Louisville, KY (US)

(21) Appl. No.: 17/945,198

(22) Filed: Sep. 15, 2022

Related U.S. Application Data
(60) Provisional application No. 63/244,506, filed on Sep. 15, 2021.

(51) Int. Cl. B29C 64/227; B33Y 10/00; B33Y 30/00; B29C 64/106; B29C 64/209; C12N 11/00

(52) U.S. Cl. CPC B29C 64/227; B29C 64/106; B29C 64/209; B33Y 10/00; B33Y 30/00; C12N 11/00; B29K 2995/0056; C12N 2513/00

(57) ABSTRACT

A 3D printing assembly, system, and method for 3D printing a biomaterial may include a robotic arm end effector and a barrel clamp assembly. The robotic arm end effector is configured to move along one or more axes of movement for 3D printing. The barrel clamp assembly is distally coupled to the robotic arm end effector and includes a barrel clamp arm and a barrel clamp. The barrel clamp arm includes a top end coupled to the robotic arm end effector and a bottom end opposite to the top end. The bottom end is angled forward with respect to the top end. The barrel clamp is coupled to the bottom end of the barrel clamp arm and is configured to receive and clamp against a distal end of a printing syringe barrel for 3D printing.

### CROSS REFERENCE TO RELATED APPLICATIONS

This application claims the benefit of U.S. Provisional Application Serial No. 63/244,506, filed Sep. 15, 2021, the entirety of which is incorporated by reference herein.

### TECHNICAL FIELD

Embodiments of the present disclosure generally relate to 3D printing tools and assemblies and, more specifically, to 3D printing tools and assemblies for clamping a distal end of a printing syringe barrel.

### BACKGROUND

Additive manufacturing apparatuses may be utilized to build an object from building material, such as organic or inorganic powders, in a layer-wise manner. Tissue engineering via 3D biomaterial dispenser-based deposition, in particular, is a fast-evolving technology. The rapid growth in the 3D tissue engineering industry is in large part due to a demand for transplantable organs and organ repair tissues that is increasing at a faster rate than the supply. Hence, the prospect for time urgent, large volume fabrication of synthetic biological constructs, including functional tissues and organs, has wide-spread appeal.

Printing syringe barrels from which to dispose biomaterial may be used in such additive and subtractive manufacturing and other applications. A need exists for more efficient and accurate printing via the printing syringe barrel when used for bioprinting.

### SUMMARY

In accordance with one embodiment of the present disclosure, a 3D printing tool and assembly for 3D printing of a biomaterial may include a robotic arm end effector and a barrel clamp assembly. The robotic arm end effector is configured to move along one or more axes of movement for 3D printing, and the barrel clamp assembly is distally coupled to the robotic arm end effector. The barrel clamp assembly includes a barrel clamp arm and a barrel clamp. The barrel clamp arm comprises a top end coupled to the robotic arm end effector and a bottom end opposite to the top end. The bottom end is angled forward with respect to the top end. The barrel clamp is coupled to the bottom end of the barrel clamp arm. The barrel clamp is configured to receive and clamp against a distal end of a printing syringe barrel for 3D printing.

In another embodiment of the present disclosure, a method for 3D printing of a biomaterial from a printing syringe barrel may include positioning a 3D printer assembly above a printing stage. The 3D printing assembly includes a robotic arm end effector and a barrel clamp assembly that is distally coupled to the robotic arm end effector. The robotic arm end effector is configured to move along one or more axes of movement for 3D printing. The barrel clamp assembly includes a barrel clamp arm and a barrel clamp. The barrel clamp arm comprises a top end coupled to the robotic arm end effector and a bottom end opposite to the top end. The bottom end is angled forward with respect to the top end. The barrel clamp is coupled to the bottom end of the barrel clamp arm, and the barrel clamp is configured to receive and clamp against a distal end of a printing syringe barrel for 3D printing. The method further may include inserting the printing syringe barrel into the barrel clamp such that the distal end of the printing syringe barrel is press fit into and clamped against by the barrel clamp, and dispensing the biomaterial from the printing syringe barrel onto the printing stage.

In yet another embodiment, a 3D printing assembly system for 3D printing of a biomaterial may include a controller, a memory communicatively coupled to the controller and storing machine-readable instructions, and a 3D printing assembly communicatively coupled to the controller. The 3D printing assembly may include a robotic arm end effector and a barrel clamp assembly that is distally coupled to the robotic arm end effector. The robotic arm end effector is configured to move along one or more axes of movement for 3D printing. The barrel clamp assembly includes a barrel clamp arm and a barrel clamp. The barrel clamp arm comprises a top end coupled to the robotic arm end effector and a bottom end opposite to the top end. The bottom end is angled forward with respect to the top end. The barrel clamp is coupled to the bottom end of the barrel clamp arm, and the barrel clamp is configured to receive and clamp against a distal end of a printing syringe barrel for 3D printing. The machine-readable instructions, when executed by the controller, may cause the 3D printing assembly to position the 3D printing assembly above a printing stage, and, after the printing syringe barrel is inserted into the barrel clamp such that the distal end of the printing syringe is press fit into and clamped against by the barrel clamp, dispense the biomaterial from the printing syringe barrel onto the printing stage.

These and additional features provided by the embodiments described herein will be more fully understood in view of the following detailed description, in conjunction with the drawings.

### BRIEF DESCRIPTION OF THE DRAWINGS

The following detailed description of specific embodiments of the present disclosure can be best understood when read in conjunction with the following drawings, in which:

FIG. 1 depicts a side-view of a 3D bioprinting assembly system including a 3D printing assembly with a barrel clamp assembly clamping against a distal end of a printing syringe barrel, according to one or more embodiments as shown and described herein;

FIG. 2 depicts a front perspective view of a 3D printing assembly of FIG. 1;

FIG. 3 depicts a bottom perspective view of the barrel clamp assembly and printing syringe barrel of FIG. 1;

FIG. 4 depicts a cross sectional view of the barrel clamp assembly and printing syringe barrel of FIG. 3 taken along line 4-4 of FIG. 3;

FIG. 5 depicts a cross sectional view of the barrel clamp assembly of FIG. 3;

FIG. 6 is a front perspective view of the 3D printing assembly of FIG. 1 with the barrel clamp assembly clamping against a smaller printing syringe barrel than that of FIG. 1; and

FIG. 7 depicts a flowchart for 3D printing of a biomaterial via use of the 3D printing assembly of FIGS. 1-6, according to one or more embodiments as shown and described herein.

### DETAILED DESCRIPTION

A 3D bioprinting system can design, fabricate and assemble complex three-dimensional biomaterial constructs, including, but not limited to, cellular systems, tissues, organs, and implantable medical devices and jigs, utilizing a 3D bioprinter. In embodiments, the bioassembly system achieves extrusion dispensing of biomaterials. Suitable materials include, but are not limited to, biomaterials such as cells, biosupport materials such as gels, and non-biological materials, such as for use in the design and fabrication of implantable jigs. Combinations of biomaterials, biosupport materials, and non-biological materials may be utilized in the same fabrication. For simplicity, as used herein “bioprinting” refers broadly to any biomaterial dispensing technology utilizing three-dimensional, precise deposition of biomaterials via methodology that is compatible with an automated, computer-aided, three-dimensional prototyping device (a bioprinter). As used herein, “biomaterial” means a liquid, semi-solid, or solid composition comprising a plurality of cells, cell solutions, cell aggregates, multicellular forms or tissues, and in all cases may include support material such as gels, hydrogels, alginate or non-cellular materials that provide specific biomechanical properties that enable biomaterial printing.

As used herein, “dispensing of biomaterials” may be effectuated by any bioprinting technique including but not limited to inkjet, extrusion/microextrusion, and laser-assisted printing. Thermal inkjet printers electrically heat the print head to produce air-pressure pulses that force droplets from the nozzle, while acoustic printers use pulses formed by piezoelectric or ultrasound pressure. Extrusion printers typically rely on pneumatic or mechanical (piston or screw) dispensing mechanisms to extrude continuous beads or filaments of biomaterial (or non-biomaterial). Laser-assisted printers use lasers focused on an absorbing substrate to generate pressures that propel cell-containing materials onto the substrate. According to embodiments, the robotically controlled bioprinting of the 3D bioprinter comprises extrusion dispensing onto a substrate using a pneumatic actuator.

Embodiments described herein are directed to a three-dimensional (“3D”) printing assembly and systems and methods for 3D printing of a biomaterial via dispense from one or more printing syringe barrels in unit tools employed in a 3D bioprinting system. A barrel clamp of the 3D printing assembly is configured to clamp a distal end of a printing syringe barrel from which the biomaterial is disposed. In embodiments, the printing syringe barrel does not require any other additional means of fastening or gripping by the 3D printing assembly. The ability to have a multitude of printing syringe barrel sizes placed in a tool rapidly without the need of extra fasteners while also minimizing the pressure induced movement of the printing syringe barrel needle can help create more efficient and rapid printing with less potential waste of time and materials. Various embodiments of a 3D printing tool and assembly for dispensing multiple materials is disclosed and detailed herein.

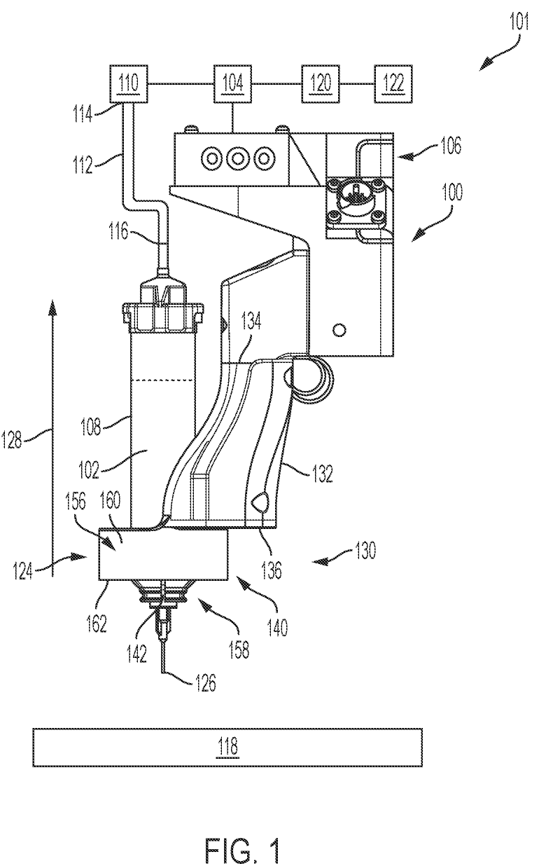

Referring to FIG. 1, a 3D printing assembly system 101 for printing of a biomaterial 102 within a printing syringe barrel 108 onto a printing stage 118. The 3D printing assembly system 101 includes a controller 120, a memory 122 storing machine-readable instructions, and a 3D printing assembly 100. The memory 122 and the 3D printing assembly 100 are communicatively coupled to the controller 120. The various components of the 3D printing assembly system 101 and the interaction thereof will be described in detail below.

The 3D printing assembly system 101 can comprise multiple servers containing one or more applications and computing devices. In some embodiments, the 3D printing assembly system 101 is implemented using a wide area network (WAN) or network 222, such as an intranet or the internet. The computing device may include digital systems and other devices permitting connection to and navigation of the network. Other 3D printing assembly system 101 variations allowing for communication between various geographically diverse components are possible. The lines depicted in FIG. 1 indicate communication rather than physical connections between the various components. The communication path shown by the lines may be formed from any medium that is capable of transmitting a signal such as, for example, conductive wires, conductive traces, optical waveguides, or the like, or from a combination of mediums capable of transmitting signals. The communication path communicatively couples the various components of 3D printing assembly system 101. As used herein, the term “communicatively coupled” means that coupled components are capable of exchanging data signals with one another such as, for example, electrical signals via conductive medium, electromagnetic signals via air, optical signals via optical waveguides, and the like.

The controller 120 may be a processor, an integrated circuit, a microchip, a computer, or any other computing device communicatively coupled to the other components of the 3D printing assembly system 101 by the communication path. Accordingly, the communication path may communicatively couple any number of controllers with one another, and allow the modules coupled to the communication path to operate in a distributed computing environment. Specifically, each of the modules can operate as a node that may send and/or receive data.

The memory 122 may be a non-transitory computer readable medium or non-transitory computer readable memory and may be configured as a nonvolatile computer readable medium. The memory 122 may comprise RAM, ROM, flash memories, hard drives, or any device capable of storing machine readable instructions such that the machine readable instructions can be accessed and executed by the controller 120. The machine readable instructions may comprise logic or algorithm(s) written in any programming language such as, for example, machine language that may be directly executed by the processor, or assembly language, object-oriented programming (OOP), scripting languages, microcode, etc., that may be compiled or assembled into machine readable instructions and stored on the memory 122. Alternatively, the machine readable instructions may be written in a hardware description language (HDL), such as logic implemented via either a field-programmable gate array (FPGA) configuration or an application-specific integrated circuit (ASIC), or their equivalents. Accordingly, the methods described herein may be implemented in any conventional computer programming language, as pre-programmed hardware elements, or as a combination of hardware and software components.

The 3D printing assembly 100 includes a robotic arm 104, a robotic arm end effector 106, and a barrel clamp assembly 130. The robotic arm end effector 106 is distally coupled to the robotic arm 104, and the barrel clamp assembly 130 is distally coupled to the robotic arm end effector 106. The robotic arm end effector 106 and the robotic arm 104 are configured to move along one or more axes of movement for 3D printing. The above components may be coupled by press fit, welds, hooks, or other fastening devices or mechanisms. In some embodiments, the robotic arm end effector may be coupled to other actuation hardware configured to move along one or more axes of movement for 3D printing.

In embodiments, and as shown in FIG. 1, the 3D printing assembly 100 may further include a pressure source 110 and an actuation fitting 112 having a proximal end 114 and a distal end 116. The pressure source 110 may be communicatively coupled to the controller 120. The proximal end 114 of the actuation fitting 112 may be configured to be coupled to the pressure source 110. The distal end 116 of the actuation fitting 112 may be configured to be coupled to the printing syringe barrel 108 to provide dispensing pressure to the printing syringe barrel 108 via the pressure source 110 to move the printing syringe barrel 108 from a resting state to a dispensing state. When the printing syringe barrel 108 is in the dispensing state, a position of a distal needle 126 coupled to a distal end 124 of the printing syringe barrel 108 may be distally displaced less than 25 micrometers (“µm”) with respect to the position of the distal needle 126 when the printing syringe barrel 108 is in the resting state. In embodiments, when the printing syringe barrel 108 is in the dispensing state, a position of a distal needle 126 coupled to a distal end 124 of the printing syringe barrel 108 may be distally displaced within a range of from about 20 µm to about 25 µm with respect to the position of the distal needle 126 when the printing syringe barrel 108 is in the resting state. The distal needle 126 is coupled to the distal end 124 of the printing syringe barrel 108 and extends in a downward direction from the distal end 124 of the printing syringe barrel 108. Biomaterial 102 may be dispensed from the distal needle 126 onto the printing stage 118 to form biomaterial constructs as described herein. When dispensing pressure is provided to the printing syringe barrel 108 via the actuation fitting 112 and the pressure source 110, a pressure induced distension in an upward proximal direction 128 may be incurred by the printing syringe barrel 108 as shown in FIG. 1. The pressure induces distension in the upward proximal direction 128 mitigates pressure-induced axial distension of the distal end 124 of the printing syringe barrel 108 such that the a position of a distal needle 126 coupled to a distal end 124 of the printing syringe barrel 108 may be distally displaced less than 25 µm in a dispensing state with respect to the position of the distal needle 126 when the printing syringe barrel 108 is in the resting state.

The barrel clamp assembly 130 includes a barrel clamp arm 132 and a barrel clamp 140. The barrel clamp arm 132 includes a top end 134 and a bottom end 136. The top end 134 is coupled to the robotic arm end effector 106, such as shown at a distal end of the robotic arm end effector 106 in FIG. 1. The bottom end 136 is opposite the top end 134. In some embodiments, the bottom end 136 is angled forward with respect to the top end 134, which, as shown in FIG. 1, may create a curved barrel clamp arm 132. The barrel clamp 140 is coupled to the bottom end 136 of the barrel clamp arm 132. As shown in FIG. 1, the barrel clamp 140 is configured to receive and clamp against the distal end 124 of the printing syringe barrel 108 for 3D printing of biomaterial 102 such as through the distal needle 126 of the printing syringe barrel 108 onto the printing stage 118.

In some embodiments, the barrel clamp 140 may be made from nylon. Additionally or alternatively, in other embodiments, the barrel clamp 140 may be made from aluminum, steel, plastic, composites, any combination thereof, or a suitable like material. The barrel clamp 140 may include a top portion 156 and a bottom portion 158. The top portion 156 includes a top surface 160 and a bottom surface 162 distally opposing the top surface 160. The bottom portion 158 of the barrel clamp 140 may include a radial array of ribs 142. The ribs 142 may assist to increase stiffness to overcome a tolerance state when the printing syringe barrel 108 is press fit against the barrel clamp 140. A greater number of ribs 142 may result in a greater compliancy with respect to the press fit. In some embodiments, bottom portion 158 of the barrel clamp 140 may instead be a radial array of gripping fingers that may assist to increase stiffness to overcome a tolerance state when the printing syringe barrel 108 is press fit against the barrel clamp 140.

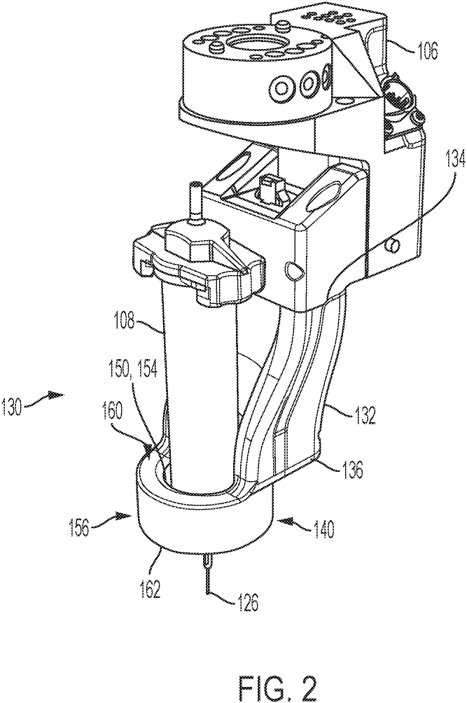

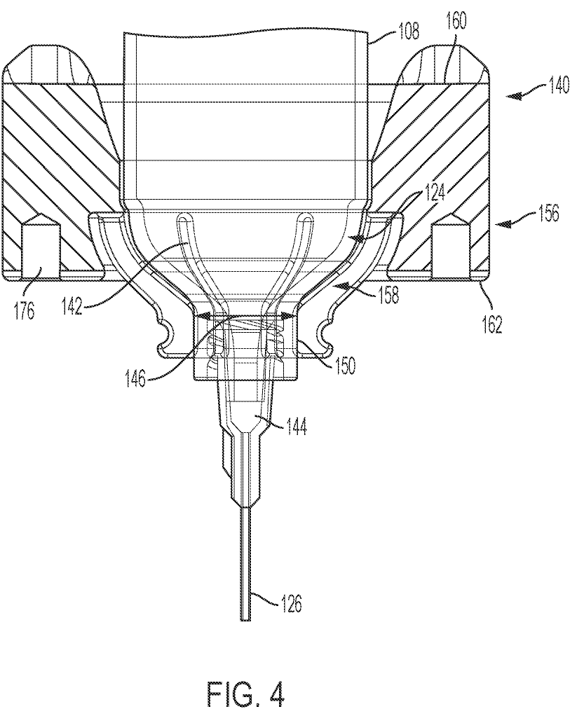

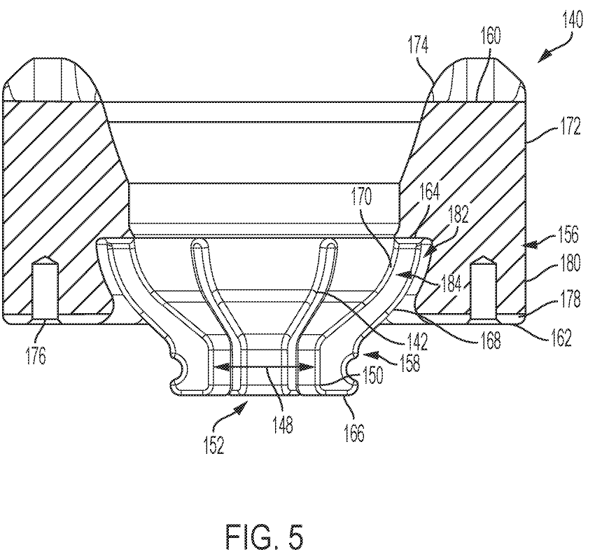

Referring to FIG. 2, a front perspective view of the 3D printing assembly 100 is shown. The barrel clamp 140 is shown as including a wall 150 defining a barrel clamp aperture 152 (FIG. 5). As shown in FIGS. 1-5, the distal end 124 of the printing syringe barrel 108 is configured to contact the wall 150 defining the barrel clamp aperture 152. In embodiments, the barrel clamp 140 may include one or more walls defining at least three points of contact, and the distal end 124 of the printing syringe barrel 108 may be configured to contact the one or more walls at the at least three points of contact. The at least three points of contact may be equidistant from each other to define a symmetrical radial clamping against the distal end 124 of the printing syringe barrel 108 when the distal end 124 of the printing syringe barrel 108 is received in the barrel clamp 140. The one or more walls defining the at least three points of contact may be a cylindrical wall 154 (FIG. 2) defining the barrel clamp aperture 152 (FIG. 5), and the distal end 124 of the printing syringe barrel 108 may be configured to contact the cylindrical wall 154 defining the barrel clamp aperture 152. In embodiments, the cylindrical wall 154 defining the barrel clamp aperture 152 may include a clamp wall diameter 148 (FIG. 5), and the distal end 124 of the printing syringe barrel 108 may include a distal end barrel diameter 146 (FIG. 4). The clamp wall diameter 148 is less than the distal end barrel diameter 146 such that the barrel clamp 140 is configured to clamp against a received distal end 124 of the printing syringe barrel 108 as shown in FIG. 4. In embodiments, the distal end 124 of the printing syringe barrel 108 may include a luer cylinder 144.

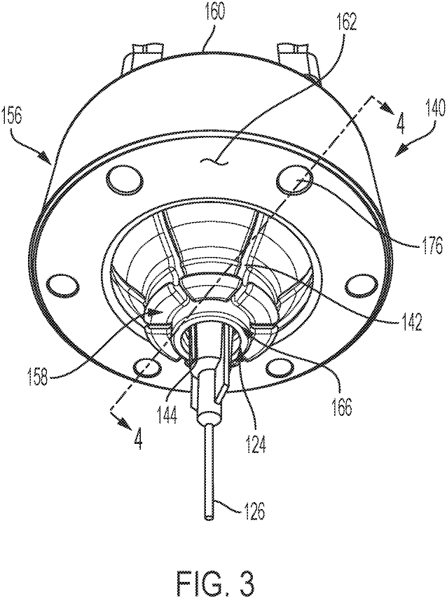

Referring to FIGS. 3-5, the bottom portion 158 of the barrel clamp 140 is configured to be flexible in order to clamp against objects by providing a uniform axisymmetric clamping and gripping force on the contacting surface of the printing syringe barrel 108. The bottom surface 162 of the top portion 156 may further include one or more fastening mechanism 176, which may additionally or alternatively include lights for ultraviolet (“UV”) curing. The bottom portion 158 of the barrel clamp 140 may include a top surface 164 and a bottom surface 166 that is distally opposed to the top surface 164.

In some embodiments, when the pressure source 110 of FIG. 1 is activated, dispensing pressure is pushed through the actuation fitting 112 and into the printing syringe barrel 108 to move the printing syringe barrel 108 from the resting state into the dispensing state. When in the dispensing state, biomaterial 102 moves through the distal needle 126 onto the printing stage 118. Further, when in the dispensing state, a pressure induced distension is incurred in a upward proximal direction 128 (FIG. 2) by the printing syringe barrel 108. The dispensing pressure in the printing syringe barrel 108 may cause the distal end 124 of the printing syringe barrel 108 to further press into and be in contact with the wall 150 defining the barrel clamp aperture 152 to provide uniform grip clamping of the distal end 124 of the printing syringe barrel 108. Such clamping may mitigate pressure-induced axial distension of the printing syringe barrel 108, thus precisely maintaining the position of the distal needle 126 of the distal end 124 of the printing syringe barrel 108. In embodiments, the pressure source 110 and actuation fitting 112 may be a pneumonic actuator.

In some embodiments, the wall 150 defining a barrel clamp aperture 152 may be a non-cylindrical shape, comprising of one or more walls with at least three points of contact. For example, a triangle, square, octagonal or other shape creating at least three points of contact. The distal end 124 of the printing syringe barrel 108 is configured to be in contact the one or more walls of the walls 150 that define the barrel clamp aperture 152 at least three points of contact.

Referring to FIG. 4, a cross sectional view of the barrel clamp 140 containing the distal end 124 of the printing syringe barrel 108 is illustrated. The top surface 160 of the top portion 156 of barrel clamp 140 is coupled to the bottom end 136 of the barrel clamp arm 132. Opposite the top surface 160 of the top portion 156 is the bottom surface 162 of the top portion 156 of the barrel clamp 140. The top portion 156 of the barrel clamp 140 contains one or more fastening mechanisms 176 that extend upward through the bottom surface 162.

Referring to FIG. 5, coupled to the top portion 156 is the bottom portion 158 of the barrel clamp 140. The bottom portion 158 comprises an outer wall 168 that tapers inward from the top surface 164 of the bottom portion 158 to the bottom surface 166 of the bottom portion 158 of the barrel clamp 140. In some embodiments, the outer wall 168 may be a non-cylindrical shape and may include one or more walls defining at least three points of contact. The bottom portion 158 may include an array of ribs 142 formed between the outer wall 168 and an inner wall 170 of the bottom portion 158 and longitudinally extending between the bottom surface 166 to the top surface 164 of the bottom portion 158. The ribs 142 may expand when press fit against the printing syringe barrel 108 about the distal end barrel diameter 146 of the distal end 124 of the printing syringe barrel 108 and provide uniform grip clamping. In some embodiments, the radial array of ribs 142 may include six equidistant ribs, though fewer and greater amounts of ribs are within the scope of this disclosure.

Referring again to the top portion 156, the top portion 156 has a top surface 160, an opposite bottom surface 162, an outer wall 172 and an inner wall 174. The inner wall 174 and the outer wall 172 extend between the top surface 160 and the bottom surface 162. The inner wall 174 defines an aperture and tapers slightly inward from the top surface 160 to the bottom surface 162, such that a diameter of the inner wall 174 at the bottom surface 162 less than a diameter of the inner wall 174 at the top surface 160. The inner wall 174 further defines a receiving portion 182 at a distal end. The top portion 156 further includes a bottom plate 178 and a top plate 180. The bottom plate 178 is coupled to the top plate 180, such as through fastening mechanisms 176.

The bottom portion 158 of the barrel clamp 140 includes the top surface 164 opposite the bottom surface 166, and the bottom portion 158 includes an outer wall 168 opposite an inner wall 170. The inner wall 170 is an embodiment of the barrel clamp wall 150 defining the barrel clamp aperture 152. The bottom portion 158 further includes an inserted portion 184 defined between the inner wall 170 and the outer wall 168. The inner wall 170 of the bottom portion 158 tapers inward, such that the diameter of the inner wall 170 at the bottom surface 166 is less than the diameter of the inner wall 170 at the top surface 164. The diameter of the inner wall 170 at the bottom surface 166 is the clamp wall diameter 148 of the barrel clamp aperture 152. In embodiments, the clamp wall diameter 148 is similar to but slightly less than the distal end barrel diameter 146 such that the wall 150 is press fit against the printing syringe barrel 108 when it is received into the barrel clamp aperture 152 of the barrel clamp 140. In some embodiments, the barrel clamp wall 150 may be a non-cylindrical shape, comprising of one or more walls 150 with at least three points of contact. For example, a triangle, square, octagonal or other shape creating at least three points of contact. In embodiments, the points of contact are equidistant from each other to define a symmetrical radial clamping against the distal end 124 of the printing syringe barrel 108.

In embodiments, the receiving portion 182 of the top portion 156 receives the inserted portion 184 of the bottom portion 158. In other embodiments, the receiving portion 182 of the top portion 156 may be a void of a different size or shape as to accommodate a different size or shape of the inserted portion 184 of the bottom portion 158 (e.g., comprising ribs 142), for example, but not limited to, a longer bottom portion 158 or a different angle of incline of the taper of the bottom portion 158. As a non-limiting example, when the top plate 180 is disconnected from the bottom plate 178, the inserted portion 184 is received into the receiving portion 182, and then the bottom plate 178 is coupled (such as via the one or more fastening mechanisms 176) to the top plate 180 to hold the bottom portion 158 against the top portion 156 of the barrel clamp 140. The fastening mechanism 176 extends through the bottom plate 178 into the top plate 180. In embodiments, the bottom portion 158 may be coupled to the top portion 156 by press fit, welds, hooks, or other fastening devices or mechanisms. Alternatively or additionally, the fastening mechanism 176 may include a light, heating, or cooling tools.

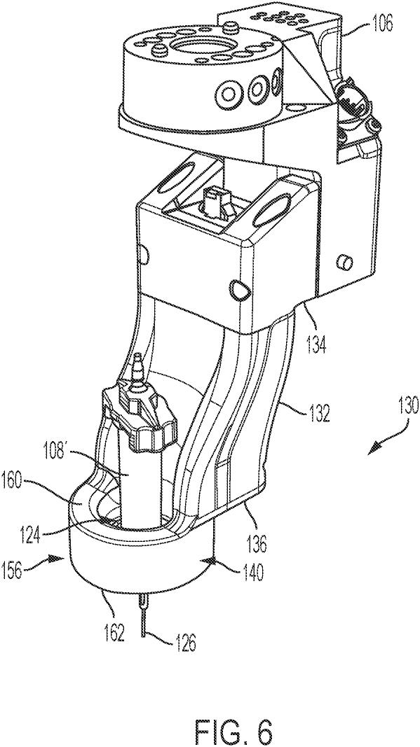

Now referring to FIG. 6, a perspective view of the barrel clamp assembly 130 of FIG. 2 is shown with a printing syringe barrel 108' of a smaller size than the printing syringe barrel 108 of FIG. 2 inserted into the barrel clamp 140. The distal end 124 of the printing syringe barrel 108' may be a luer cylinder 144. By way of example, and not as a limitation, volume sizes of the printing syringe barrel 108, 108' may be 3, 5, 10, 30, or 50 cubic centimeters (cc). Thus, the printing syringe barrel 108 of FIG. 2 may be a 50 cc syringe while the printing syringe barrel 108' of FIG. 6 may be a 3 cc syringe.

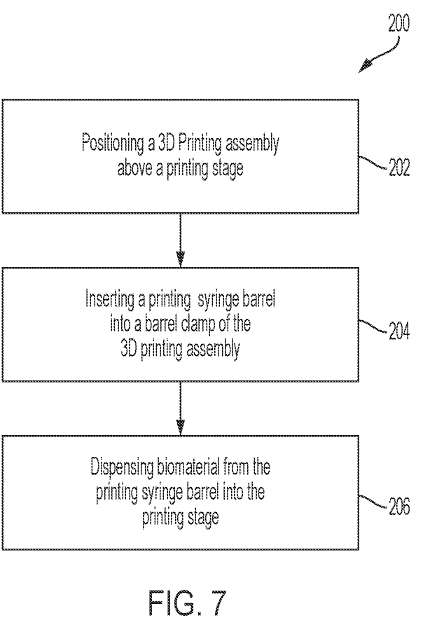

Referring now to FIG. 7, a flow diagram of a process 200 is shown that depicts a method of 3D printing a biomaterial 102 from a printing syringe barrel 108. In block 202, the 3D printing assembly 100 as described herein is positioned above the printing stage 118 (FIG. 1). In block 204, the printing syringe barrel 108, 108' as described herein is inserted into the barrel clamp 140 such that the distal end 124 of the printing syringe barrel 108, 108' is press fit into and clamped against the barrel clamp 140. In block 206, the biomaterial 102 is dispensed from the printing syringe barrel 108, 108' onto the printing stage 118. Dispensing pressure may be based on parameters such as length and diameter of the printing syringe barrel 108, 108' and may, in embodiments, between in a range of between about 0 pounds per square inch (“psi”) to 80 psi. By way of example, and not as a limitation, dispensing pressure is provided to the printing syringe barrel 108, 108' via the pressure source 110 to move the printing syringe barrel 108, 108' from the resting state to the dispensing state while the pressure induced distension is incurred in the upward proximal direction 128 by the printing syringe barrel 108, 108' based on the provided dispensing pressure. When the printing syringe barrel 108 is in the dispensing state, a position of the distal needle 126 coupled to the distal end 124 of the printing syringe barrel 108, 108' may be distally displaced such as less than 25 µm with respect to the position of the distal needle 126 when the printing syringe barrel 108, 108' is in the resting state. Unless otherwise defined, all technical and scientific terms used herein have the same meaning as commonly understood by one of ordinary skill in the art to which this disclosure belongs. All publications, patent applications, patents, and other references mentioned herein are incorporated by reference in their entirety.

### Aspects Listing

Aspect 1. A 3D printing tool and assembly for 3D printing of a biomaterial includes a robotic arm end effector and a barrel clamp assembly. The robotic arm end effector is configured to move along one or more axes of movement for 3D printing, and the barrel clamp assembly is distally coupled to the robotic arm end effector. The barrel clamp assembly includes a barrel clamp arm and a barrel clamp. The barrel clamp arm comprises a top end coupled to the robotic arm end effector and a bottom end opposite to the top end. The bottom end is angled forward with respect to the top end. The barrel clamp is coupled to the bottom end of the barrel clamp arm. The barrel clamp is configured to receive and clamp against a distal end of a printing syringe barrel for 3D printing.

Aspect 2. The 3D printing assembly of Aspect 1, wherein the barrel clamp comprises a wall defining a barrel clamp aperture, and the distal end of the printing syringe barrel is configured to contact the wall defining the barrel clamp aperture.

Aspect 3. The 3D printing assembly of Aspect 2, wherein the wall defining the barrel clamp aperture comprises a clamp wall diameter, the distal end of the printing syringe barrel comprises a distal end barrel diameter, and the clamp wall diameter is less than the distal end barrel diameter.

Aspect 4. The 3D printing assembly of any of Aspect 1 to Aspect 3, wherein the distal end of the printing syringe barrel comprises a luer cylinder.

Aspect 5. The 3D printing assembly of any of Aspect 1 to Aspect 4, wherein the barrel clamp comprises one or more walls defining at least three points of contact, and the distal end of the printing syringe barrel is configured to contact the one or more walls at the at least three points of contact.

Aspect 6. The 3D printing assembly of Aspect 5, wherein the at least three points of contact are equidistant from each other to define a symmetrical radial clamping against the distal end of the printing syringe barrel when the distal end of the printing syringe barrel is received in the barrel clamp.

Aspect 7. The 3D printing assembly of Aspect 5 or Aspect 6, wherein the one or more walls defining the at least three points of contact comprise a cylindrical wall defining a barrel clamp aperture, and the distal end of the printing syringe barrel is configured to contact the cylindrical wall defining the barrel clamp aperture.

Aspect 8. The 3D printing assembly of any of Aspect 1 to Aspect 7, further comprising a pressure source, and an actuation fitting comprising a proximal end and a distal end, the proximal end of the actuation fitting configured to be coupled to the pressure source, and the distal end of the actuation fitting configured to be coupled to the printing syringe barrel to provide dispensing pressure to the printing syringe barrel via the pressure source to move the printing syringe barrel from a resting state to a dispensing state.

Aspect 9. The 3D printing assembly of Aspect 8, wherein when the printing syringe barrel is in the dispensing state, a position of a distal needle coupled to the distal end of the printing syringe barrel is distally displaced less than 25 µm with respect to the position of the distal needle when the printing syringe barrel is in the resting state.

Aspect 10. The 3D printing assembly of Aspect 8 or Aspect 9, wherein when dispensing pressure is provided to the printing syringe barrel via the actuation fitting and the pressure source, a pressure induced distension in an upward proximal direction is incurred by the printing syringe barrel.

Aspect 11. The 3D printing assembly of any of Aspect 8 to Aspect 10, wherein a wall defining a barrel clamp aperture of the barrel clamp comprises a radial array of ribs configured to clamp against the distal end of the printing syringe barrel.

Aspect 12. The 3D printing assembly of any of Aspect 1 to Aspect 11, wherein the barrel clamp comprises nylon, aluminum, steel, or combinations thereof.

Aspect 13. A method for 3D printing of a biomaterial from a printing syringe barrel includes positioning a 3D printer assembly above a printing stage. The 3D printing assembly includes a robotic arm end effector and a barrel clamp assembly that is distally coupled to the robotic arm end effector. The robotic arm end effector is configured to move along one or more axes of movement for 3D printing. The barrel clamp assembly includes a barrel clamp arm and a barrel clamp. The barrel clamp arm comprises a top end coupled to the robotic arm end effector and a bottom end opposite to the top end. The bottom end is angled forward with respect to the top end. The barrel clamp is coupled to the bottom end of the barrel clamp arm, and the barrel clamp is configured to receive and clamp against a distal end of a printing syringe barrel for 3D printing. The method further may include inserting the printing syringe barrel into the barrel clamp such that the distal end of the printing syringe barrel is press fit into and clamped against by the barrel clamp, and dispensing the biomaterial from the printing syringe barrel onto the printing stage.

Aspect 14. The method of Aspect 13, further comprising contacting the distal end of the printing syringe barrel against a wall defining a barrel clamp aperture of the barrel clamp.

Aspect 15. The method of Aspect 14, wherein the wall defining the barrel clamp aperture comprises a clamp wall diameter, the distal end of the printing syringe barrel comprises a distal end barrel diameter, and the clamp wall diameter is less than the distal end barrel diameter.

Aspect 16. The method of any of Aspect 13 to Aspect 15, wherein the distal end of the printing syringe barrel comprises a luer cylinder.

Aspect 17. The method of any of Aspect 13 to Aspect 15, wherein the 3D printing assembly further comprises a pressure source and an actuation fitting comprising a proximal end and a distal end, the proximal end of the actuation fitting configured to be coupled to the pressure source, and the distal end of the actuation fitting configured to be coupled to the printing syringe barrel to provide dispensing pressure to the printing syringe barrel via the pressure source to move the printing syringe barrel from a resting state to a dispensing state.

Aspect 18. The method of Aspect 17, wherein the method further comprises providing dispensing pressure to the printing syringe barrel via the pressure source to move the printing syringe barrel from the resting state to the dispensing state, incurring a pressure induced distension in an upward proximal direction by the printing syringe barrel based on the provided dispensing pressure, and when the printing syringe barrel is in the dispensing state, distally displacing a position of a distal needle coupled to the distal end of the printing syringe barrel less than 25 µm with respect to the position of the distal needle when the printing syringe barrel is in the resting state.

Aspect 19. A 3D printing assembly system for 3D printing of a biomaterial may include a controller, a memory communicatively coupled to the controller and storing machine-readable instructions, and a 3D printing assembly communicatively coupled to the controller. The 3D printing assembly may include a robotic arm end effector and a barrel clamp assembly that is distally coupled to the robotic arm end effector. The robotic arm end effector is configured to move along one or more axes of movement for 3D printing. The barrel clamp assembly includes a barrel clamp arm and a barrel clamp. The barrel clamp arm comprises a top end coupled to the robotic arm end effector and a bottom end opposite to the top end. The bottom end is angled forward with respect to the top end. The barrel clamp is coupled to the bottom end of the barrel clamp arm, and the barrel clamp is configured to receive and clamp against a distal end of a printing syringe barrel for 3D printing. The machine-readable instructions, when executed by the controller, may cause the 3D printing assembly to position the 3D printing assembly above a printing stage, and, after the printing syringe barrel is inserted into the barrel clamp such that the distal end of the printing syringe is press fit into and clamped against by the barrel clamp, dispense the biomaterial from the printing syringe barrel onto the printing stage.

Aspect 20. The 3D printing assembly system of Aspect 19, wherein the machine-readable instructions further cause the 3D printing assembly system to provide dispensing pressure to the printing syringe barrel via a pressure source to move the printing syringe barrel from a resting state to a dispensing state. The 3D printing assembly further comprises an actuation fitting comprising a proximal end and a distal end, the proximal end of the actuation fitting coupled to the pressure source, and the distal end of the actuation fitting coupled to the printing syringe barrel to provide dispensing pressure to the printing syringe barrel via the pressure source to move the printing syringe barrel from the resting state to the dispensing state. The machine-readable instructions further cause the 3D printing assembly system to incur a pressure induced distension in an upward proximal direction by the printing syringe barrel based on the provided dispensing pressure, and, when the printing syringe barrel is in the dispensing state, distally displace a position of a distal needle coupled to the distal end of the printing syringe barrel less than 25 µm with respect to the position of the distal needle when the printing syringe barrel is in the resting state.

As used in this specification and the appended claims, the singular forms “a,” “an,” and “the” include plural references unless the context clearly dictates otherwise. Any reference to “or” herein is intended to encompass “and/or” unless otherwise stated.

Specific embodiments will now be described with references to the figures. Whenever possible, the same reference numerals will be used throughout the drawings to refer to the same or like parts.

It should be apparent to those skilled in the art that various modifications and variations may be made to the embodiments described within without departing from the spirit and scope of the claimed subject matter. Thus, it is intended that the specification cover the modifications and variations of the various embodiments described within provided such modification and variations come within the scope of the appended claims and their equivalents.

Having described the subject matter of the present disclosure in detail and by reference to specific embodiments thereof, it is noted that the various details disclosed within should not be taken to imply that these details relate to elements that are essential components of the various embodiments described within, even in cases where a particular element is illustrated in each of the drawings that accompany the present description. Further, it should be apparent that modifications and variations are possible without departing from the scope of the present disclosure, including, but not limited to, embodiments defined in the appended claims. More specifically, although some aspects of the present disclosure are identified as particularly advantageous, it is contemplated that the present disclosure is not necessarily limited to these aspects.

What is claimed is:

1. A 3D printing assembly for 3D printing of a biomaterial, the 3D printing assembly comprising:
a robotic arm end effector configured to move along one or more axes of movement for 3D printing; and
a barrel clamp assembly distally coupled to the robotic arm end effector, the barrel clamp assembly comprising:
a barrel clamp arm comprising a top end coupled to the robotic arm end effector and a bottom end opposite the top end, wherein the bottom end is angled forward with respect to the top end; and
a barrel clamp, wherein the barrel clamp is coupled to the bottom end of the barrel clamp arm, and wherein the barrel clamp is configured to receive and clamp against a distal end of a printing syringe barrel for 3D printing.

2. The 3D printing assembly of claim 1, wherein the barrel clamp comprises a wall defining a barrel clamp aperture, and the distal end of the printing syringe barrel is configured to contact the wall defining the barrel clamp aperture.

3. The 3D printing assembly of claim 2, wherein the wall defining the barrel clamp aperture comprises a clamp wall diameter, the distal end of the printing syringe barrel comprises a distal end barrel diameter, and the clamp wall diameter is less than the distal end barrel diameter.

4. The 3D printing assembly of claim 1, wherein the distal end of the printing syringe barrel comprises a luer cylinder.

5. The 3D printing assembly of claim 1, wherein the barrel clamp comprises one or more walls defining at least three points of contact, and the distal end of the printing syringe barrel is configured to contact the one or more walls at the at least three points of contact.

6. The 3D printing assembly of claim 5, wherein the at least three points of contact are equidistant from each other to define a symmetrical radial clamping against the distal end of the printing syringe barrel when the distal end of the printing syringe barrel is received in the barrel clamp.

7. The 3D printing assembly of claim 5, wherein the one or more walls defining the at least three points of contact comprise a cylindrical wall defining a barrel clamp aperture, and the distal end of the printing syringe barrel is configured to contact the cylindrical wall defining the barrel clamp aperture.

8. The 3D printing assembly of claim 1, further comprising:
a pressure source; and
an actuation fitting comprising a proximal end and a distal end, the proximal end of the actuation fitting configured to be coupled to the pressure source, and the distal end of the actuation fitting configured to be coupled to the printing syringe barrel to provide dispensing pressure to the printing syringe barrel via the pressure source to move the printing syringe barrel from a resting state to a dispensing state.

9. The 3D printing assembly of claim 8, wherein when the printing syringe barrel is in the dispensing state, a position of a distal needle coupled to the distal end of the printing syringe barrel is distally displaced less than 25 µm with respect to the position of the distal needle when the printing syringe barrel is in the resting state.

10. The 3D printing assembly of claim 8, wherein when dispensing pressure is provided to the printing syringe barrel via the actuation fitting and the pressure source, a pressure induced distension in an upward proximal direction is incurred by the printing syringe barrel.

11. The 3D printing assembly of claim 8, wherein a wall defining a barrel clamp aperture of the barrel clamp comprises a radial array of ribs configured to clamp against the distal end of the printing syringe barrel.

12. The 3D printing assembly of claim 1, wherein the barrel clamp comprises nylon, aluminum, steel, or combinations thereof.

13. A method for 3D printing of a biomaterial from a printing syringe barrel, the method comprising:
positioning a 3D printing assembly above a printing stage, the 3D printing assembly comprising:
a robotic arm end effector configured to move along one or more axes of movement for 3D printing; and
a barrel clamp assembly distally coupled to the robotic arm end effector, the barrel clamp assembly comprising:
a barrel clamp arm comprising a top end coupled to the robotic arm end effector and a bottom end opposite the top end, wherein the bottom end is angled forward with respect to the top end; and
a barrel clamp, wherein the barrel clamp is coupled to the bottom end of the barrel clamp arm, and wherein the barrel clamp is configured to receive and clamp against a distal end of the printing syringe barrel for 3D printing;
inserting the printing syringe barrel into the barrel clamp such that the distal end of the printing syringe barrel is press fit into and clamped against the barrel clamp; and
dispensing the biomaterial from the printing syringe barrel onto the printing stage.

14. The method of claim 13, further comprising contacting the distal end of the printing syringe barrel against a wall defining a barrel clamp aperture of the barrel clamp.

15. The method of claim 14, wherein the wall defining the barrel clamp aperture comprises a clamp wall diameter, the distal end of the printing syringe barrel comprises a distal end barrel diameter, and the clamp wall diameter is less than the distal end barrel diameter.

16. The method of claim 13, wherein the distal end of the printing syringe barrel comprises a luer cylinder.

17. The method of claim 13, wherein the 3D printing assembly further comprises:
a pressure source; and
an actuation fitting comprising a proximal end and a distal end, the proximal end of the actuation fitting configured to be coupled to the pressure source, and the distal end of the actuation fitting configured to be coupled to the printing syringe barrel to provide dispensing pressure to the printing syringe barrel via the pressure source to move the printing syringe barrel from a resting state to a dispensing state.

18. The method of claim 17, wherein the method further comprises:
providing dispensing pressure to the printing syringe barrel via the pressure source to move the printing syringe barrel from the resting state to the dispensing state;
incurring a pressure induced distension in an upward proximal direction by the printing syringe barrel based on the provided dispensing pressure; and
when the printing syringe barrel is in the dispensing state, distally displacing a position of a distal needle coupled to the distal end of the printing syringe barrel less than 25 µm with respect to the position of the distal needle when the printing syringe barrel is in the resting state.

19. A 3D printing assembly system for 3D printing of a biomaterial, the 3D printing assembly system comprising:
a controller;
a memory communicatively coupled to the controller and storing machine-readable instructions; and
a 3D printing assembly communicatively coupled to the controller, the 3D printing assembly comprising:
a robotic arm end effector configured to move along one or more axes of movement for 3D printing; and
a barrel clamp assembly distally coupled to the robotic arm end effector, the barrel clamp assembly comprising:
a barrel clamp arm comprising a top end coupled to the robotic arm end effector and a bottom end opposite the top end, wherein the bottom end is angled forward with respect to the top end; and
a barrel clamp, wherein the barrel clamp is coupled to the bottom end of the barrel clamp arm, and wherein the barrel clamp is configured to receive and clamp against a distal end of a printing syringe barrel for 3D printing
wherein the machine-readable instructions, when executed by the controller, cause the 3D printing assembly system to:
position the 3D printing assembly above a printing stage; and
after the printing syringe barrel is inserted into the barrel clamp such that the distal end of the printing syringe barrel is press fit into and clamped against the barrel clamp, dispense the biomaterial from the printing syringe barrel onto the printing stage.

20. The 3D printing assembly system of claim 19, wherein the machine-readable instructions further cause the 3D printing assembly system to:
provide dispensing pressure to the printing syringe barrel via a pressure source to move the printing syringe barrel from a resting state to a dispensing state, wherein the 3D printing assembly further comprises an actuation fitting comprising a proximal end and a distal end, the proximal end of the actuation fitting coupled to the pressure source, and the distal end of the actuation fitting coupled to the printing syringe barrel to provide dispensing pressure to the printing syringe barrel via the pressure source to move the printing syringe barrel from the resting state to the dispensing state;
incur a pressure induced distension in an upward proximal direction by the printing syringe barrel based on the provided dispensing pressure; and
when the printing syringe barrel is in the dispensing state, distally displace a position of a distal needle coupled to the distal end of the printing syringe barrel less than 25 µm with respect to the position of the distal needle when the printing syringe barrel is in the resting state.
